(19) United States
(12) Patent Application Publication
(10) Pub. No.: US 2024/0168038 A1
(43) Pub. Date: May 23, 2024

(54) METHOD OF DETERMINING PROGNOSIS OF CHRONIC KIDNEY DISEASE

(71) Applicants: RANDOX LABORATORIES, Crumlin Antrim (GB); RANDOX TEORANTA, County Donegal Dungloe (IE)

(72) Inventors: Ivan McConnell, Crumlin Antrim (GB); Peter Fitzgerald, Crumlin Antrim (GB); John Lamont, Crumlin Antrim (GB); Ciaran Richarson, County Donegal Dungloe (IE); Matthew Griffin, Galway (IE)

(21) Appl. No.: 18/283,652

(22) PCT Filed: Mar. 25, 2022
(86) PCT No.: PCT/EP2022/057927

§ 371 Date: Sep. 22, 2023

(30) Foreign Application Priority Data
Mar. 26, 2021 (GB) 2104287.4

(51) Int. Cl. G01N 33/68

(52) U.S. Cl. CPC G01N 33/6893; G01N 2333/4716; G01N 2333/70578; G01N 2800/347; G01N 2800/52

(57) ABSTRACT

The current invention provides methods which can be implemented alongside clinical variables as risk prediction tools for improved prediction of CKD progression and mortality. Surprisingly it was found that decreased C3a-desArg was associated with adverse outcome in CKD, and when combined with increased STNFR1 and NGAL it gave an excellent predictor of progression to a composite endpoint in CKD patients.

### BACKGROUND

Chronic kidney disease (CKD) is a growing public health problem, with its prevalence increasing by 29.3% since 1990 to affect 9.1% of the global population in 2017 (1). Over the same timeframe, mortality rates from CKD increased by 41.5% such that an estimated 1.2 million people died from CKD in 2017 (1). Although age-standardised morbidity and mortality rates from other non-communicable diseases including cardiovascular disease have declined over the past 3 decades, no such favourable trends exist for CKD (2). CKD is associated with a range of adverse outcomes, including progression of kidney disease, cardiovascular disease, and early mortality. Death due to cardiovascular disease is over-represented amongst people with CKD and mortality rates increase as estimated glomerular filtration rate (eGFR) declines. In a meta-analysis of 21 general population cohorts incorporating over 1.2 million participants, eGFR independently predicted mortality risk in an almost linear fashion (3).

Communicating risk of adverse outcomes to patients with CKD is challenging, particularly at earlier, typically asymptomatic disease stages. Additionally, while conventional tools for CKD staging and prognostication, including eGFR and urine albumin-to-creatinine ratio (uACR), are strongly predictive of adverse outcomes at the population level, their prognostic value is weaker at the individual patient level where significant variability in risks of adverse outcomes exists. Circulating biomarkers hold promise as a means of more accurately identifying patients with CKD at higher risk of adverse renal and cardiovascular outcomes who may benefit from intensification of therapy. Indeed, strong independent associations between several circulating biomarkers and risk of adverse outcomes has been demonstrated in people with CKD with and without type 2 diabetes—most notably proteins involved in inflammation cascades including members of the tumour necrosis factor (TNF) superfamily (4, 5).

Measuring multiple circulating biomarkers simultaneously has the potential to uncover subgroups of patients with CKD who have differing risks of progressive renal functional decline and mortality, although assimilation of the predictive value provided by multiple biomarkers to meaningfully communicate risks of adverse outcomes remains a challenge. Additionally, many biomarker studies to date have enrolled specific subgroups of patients with CKD, for example diabetic kidney disease; predictive performance of circulating biomarkers across the spectrum of CKD severity and aetiology in real-world outpatient nephrology practice is underexplored. EP3052939 demonstrated that complement 3a des-arginine (C3a-desArg) was elevated in patients with early stage CKD and could be used as a diagnostic thereof in combination with other biomarkers. Likewise, Ozata et al 2002 (6) and Tang et al 2008 (7) found elevated C3a-DesArg (also known as acylation-stimulating protein) in patients with nephrotic syndrome and paediatric proteinuric renal disease respectively. Herein, we report on the ability of a panel of serum biomarkers to predict renal and mortality endpoints over 4-5-year follow-up in 139 people with a broad range of CKD aetiologies and severity attending a tertiary referral nephrology centre. Surprisingly it was found that decreased C3a-desArg was associated with adverse outcome in CKD, and when combined with increased STNFR1 and NGAL it gave an excellent predictor of progression to a composite endpoint in CKD patients.

### REFERENCES

1. GBD Chronic Kidney Disease Collaboration: Global, regional, and national burden of chronic kidney disease, 1990-2017: a systematic analysis for the Global Burden of Disease Study 2017. Lancet, 395: 709-733, 2020. D01:10.1016/50140-6736(20)30045-3.
2. Jager K J, Fraser SDS: The ascending rank of chronic kidney disease in the global burden of disease study. Nephrology Dialysis Transplantation, 32: ii121-ii128, 2017. D O 1:10.1093/ndt/gfw330.
3. Matsushita K, van der Velde M, Astor B C, Woodward M, Levey A S, de Jong P E, Coresh J, Gansevoort R T: Association of estimated glomerular filtration rate and albuminuria with all-cause and cardiovascular mortality in general population cohorts: a collaborative meta-analysis. Lancet (London, England), 375: 2073-2081, 2010. D01:10.1016/S0140-6736(10)60674-5.
4. Niewczas M A, Pavkov M E, Skupien J, Smiles A, Md Dom Z I, Wilson J M, Park J, Nair V, Schlafly A, Saulnier P J, Satake E, Simeone C A, Shah H, Qiu C, Looker H C, Fiorina P, Ware C F, Sun J K, Doria A, Kretzler M, Susztak K, Duffin K L, Nelson R G, Krolewski A S: A signature of circulating inflammatory proteins and development of end-stage renal disease in diabetes. Nature medicine, 25: 805-813, 2019. D01:10.1038/s41591-019-0415-5.
5. Niewczas M A, Gohda T, Skupien J, Smiles A M, Walker W H, Rosetti F, Cullere X, Eckfeldt J H, Doria A, Mayadas T N, Warram J H, Krolewski A S: Circulating TNF Receptors 1 and 2 Predict ESRD in Type 2 Diabetes. Journal of the American Society of Nephrology, 23: 507, 2012. DOI: 10.1681/ASN.2011060627.
6. Metin Ozata, Cagatay Oktenli, Mustafa Gulec, Taner Ozgurtas, Fatih Bulucu, Kayser Cagier, Necati Bingol, Abdulgaffar Vural, I. Caglayan Ozdemir, Increased Fasting Plasma Acylation-Stimulating Protein Concentrations in Nephrotic Syndrome, The Journal of Clinical Endocrinology & Metabolism, 87(2):853-858,2002. DOI: 10.1210/jcem 0.87.2.8243.
7. Tang J H, Wen Y, Wu F, Zhao X Y, Zhang M X, Mi J, Cianflone K. Increased plasma acylation-stimulating protein in pediatric proteinuric renal disease. Pediatr Nephrol. 2008 Jun.;23(6):959-64. doi: 10.1007/s00467-007-0738-1.
8. Naicker S D, Cormican S, Griffin T P, Maretto S, Martin W P, Ferguson J P, Cotter D, Connaughton E P, Dennedy M C, Griffin M D: Chronic Kidney Disease Severity Is Associated With Selective Expansion of a Distinctive Intermediate Monocyte Subpopulation. Frontiers in Immunology, 9, 2018. D01:10.3389/fimmu.2018.02845.
9. Kolde R: pheatmap: Pretty Heatmaps. R package version 1.0.12. https://CRAN.R-project.org/package=pheatmap. 2019.
10. Kassambara A, Mundt F: factoextra: Extract and Visualize the Results of Multivariate Data Analyses. R package version 1.0.7. https://CRAN.R-project.org/package=factoextra. 2020.
11. Therneau T, Atkinson B: rpart: Recursive Partitioning and Regression Trees. R package version 4.1-15. https://CRAN.R-project.org/package=rpart. 2019.
12. Borkovec M, Madin N: ggparty: ‘ggplot’ Visualizations for the ‘partykit’ Package. R package version 1.0.0. https://CRAN.R-project.org/package=ggparty. 2019.
13. Liaw A, Wiener M: Classification and Regression by randomForest. R News 2(3), 18-22, 2002.
14. Venables W N, Ripley B D: Modern Applied Statistics with S. Fourth Edition. Springer, New York. ISBN 0-387-95457-0, 2002.
15. Therneau T M: A Package for Survival Analysis in R. R package version 3.1-12. https://CRAN.R-project.org/package=survival. 2020.
16. Kassambara A, Kosinski M, Biecek P: survminer: Drawing Survival Curves using ‘ggplot2’. R package version 0.4.6. https://CRAN.R-project.org/package=survminer. 2019.
17. Kuznetsova A, Brockhoff P B, Christensen RHB: ImerTest Package: Tests in Linear Mixed Effects Models. 2017, 82: 26, 2017. D01:10.18637/jss.v082.i13.
18. Solt F, Hu Y: dotwhisker: Dot-and-Whisker Plots of Regression Results. R package version 0.5.0. https://CRAN.R-project.org/package=dotwhisker. 2018.
19. Weaver R G, James M T, Ravani P, Weaver CGW, Lamb E J, Tonelli M, Manns B J, Quinn R R, Jun M, Hemmelgarn B R: Estimating Urine Albumin-to-Creatinine Ratio from Protein-to-Creatinine Ratio: Development of Equations using Same-Day Measurements. Journal of the American Society of Nephrology, 31: 591-601, 2020. DOI: 10.1681/asn.2019060605.

### SUMMARY OF THE INVENTION

The current invention provides methods which can be implemented alongside clinical variables as risk prediction tools for improved prediction of CKD progression and mortality. These tools may have particular use, in terms of both clinical value and cost-effectiveness, in the CKD outpatient setting.

A first aspect of the current invention is a method for determining the prognosis of chronic kidney disease (CKD) in a patient suffering therefrom, said method comprising a) determining the amount of complement 3a des-arginine (C3a-desArg) in an ex vivo sample obtained from said patient and b) determining whether the amount of C3a-desArg is altered compared to a reference value. A decreased level of C3a-desArg in a sample compared to a reference value is indicative of an adverse outcome.

A second aspect of the current invention is a method for determining the prognosis of chronic kidney disease (CKD) in a patient suffering therefrom, said method comprising a) determining the amount of soluble tumour necrosis factor receptor 1 (sTNFR1), neutrophil gelatinase-associated lipocalin (NGAL) and complement 3a des-arginine (C3a-desArg) in an ex vivo sample obtained from said patient and b) establishing the significance of the level of the biomarkers by inputting each of the biomarker concentration values into a statistical methodology to produce an output value that indicates CKD prognosis for the patient. In such methods an increased level of sTNFR1 in a sample obtained from a subject compared to a reference value in combination with an increased level of NGAL in a sample obtained from a subject compared to a reference value and a decreased level of C3a-desArg in a sample obtained from a subject compared to a reference value is most indicative of an adverse outcome.

A third aspect of the current invention is the use of a substrate comprising a probe which binds specifically to sTNFR1, a probe which binds specifically to NGAL and a probe which binds specifically to C3a-desArg to screen for prognosis of CKD in subjects suffering therefrom.

### BRIEF DESCRIPTION OF THE FIGURES

FIG. 1—A decision tree classification of the composite endpoint by serum biomarkers in the study cohort. The decision tree highlights the predictive value of simultaneously assessing multiple serum biomarkers. In this decision tree, the 3 biomarkers are ranked by their proximate level of importance to correct classification of the composite endpoint, from sTNFR1 (highest) to C3a-desArg (lowest).

Individuals with low sTNFR1 values (<3 ng/mL) had a relatively low risk of the composite endpoint (12.3%). However, not all individuals with high sTNFR1 values (≥3 ng/mL) had the same risk of the composite endpoint: those with low NGAL values (<156 ng/mL) or high NGAL values (≥156 ng/mL) coupled with high C3a-desArg values (≥2,368 ng/mL) had risks of the composite endpoint in the order of 38.9% and 43.8%, respectively. Conversely, individuals with the triad of high sTNFR1 (≥3 ng/mL), high NGAL (≥156 ng/mL), and low C3a-desArg (<2,368 ng/mL), which accounted for over 20% of the study cohort, almost universally (96.3%) developed the composite endpoint during follow-up.

FIG. 2—A Violin plot of area under the curve (AUC) values derived from 3 types of random forest classification models of the composite endpoint: clinical variables alone (age, gender, diabetes, and baseline eGFR) (light orange), serum biomarkers alone (orange), and clinical variables plus serum biomarkers (dark orange). The plot illustrates incremental improvements in correct prediction of the composite endpoint across the 3 model types.

A training set consisting of 75% of the study cohort was randomly sampled, upon which the random forest model was trained. The model was subsequently tested on the remaining 25% of the cohort, which served as the previously unseen test set, and an AUC value for classification of the composite endpoint by the model was calculated. This process was repeated 1,000 times for each model type (clinical variables, biomarkers, clinical variables plus biomarkers) with repeated random sampling of training and test sets in each iteration. Each dot on the plot represents an AUC value from a single iteration of this process.

Mean AUC values from 1,000 iterations for the 3 model types are printed on the plot: 0.78 for clinical variables alone, 0.82 for serum biomarkers alone, and 0.84 for clinical variables plus serum biomarkers. Comparisons between the 3 models are made by ANOVA, with post-hoc testing by multiplicity-corrected independent samples t-tests (all p<0.001).

FIG. 3—A loadings plot from principal components analysis reveals the influential biomarkers which drive the shifts in biomarker expression across CKD stages and when stratified by the composite endpoint. Individuals with advanced CKD who developed the composite endpoint had higher expression of biomarkers in the right of the plot, including sTNFR1, sTNFR2, NGAL, and cystatin C. Individuals with earlier stage CKD who did not develop the composite endpoint had higher expression of protective factors in the upper left corner of the plot including C3a-desArg and epidermal growth factor.

C3a-desArg, IL-8 and EGF had inverse relationships with the composite endpoint, NGAL, Cystatin C, sTNFR1 and sTNFR2 had strong positive relationships with the composite endpoint while MIP-1-alpha, CRP, FABP1 and D-dimer had modest positive relationships with the composite endpoint. The shape of the points represents statistical significance of the relationship between biomarkers and the composite endpoint by univariate logistic regression (circle=statistically significant, triangle=not statistically significant). The x- and y-axes represent principal components 1 and 2, respectively.

Composite endpoint: ≥40°/o decrease in CKD-EPI eGFR, doubling of serum creatinine, renal replacement therapy, or mortality.

### DETAILED DESCRIPTION

The present invention provides a method for determining the prognosis of chronic kidney disease (CKD) in a patient suffering therefrom, said method comprising a) determining the amount of complement 3a des-arginine (C3a-desArg) in an ex vivo sample obtained from said patient and b) determining whether the amount of C3a-desArg is altered compared to a reference value. A decreased level of C3a-desArg in a sample obtained from a subject compared to a reference value is indicative of an adverse outcome.

In a further embodiment the invention provides a method for determining the prognosis of chronic kidney disease (CKD) in a patient suffering therefrom, said method comprising a) determining the amount of soluble tumour necrosis factor receptor 1 (sTNFR1), neutrophil gelatinase-associated lipocalin (NGAL) and complement 3a des-arginine (C3a-desArg) in an ex vivo sample obtained from said patient and b) establishing the significance of the level of the biomarkers by inputting each of the biomarker concentration values into a statistical methodology to produce an output value that indicates CKD prognosis for the patient. An increased level of sTNFR1 in a sample obtained from a subject compared to a reference value in combination with an increased level of NGAL in a sample obtained from a subject compared to a reference value and a decreased level of C3a-desArg in a sample obtained from a subject compared to a reference value is indicative of an adverse outcome.

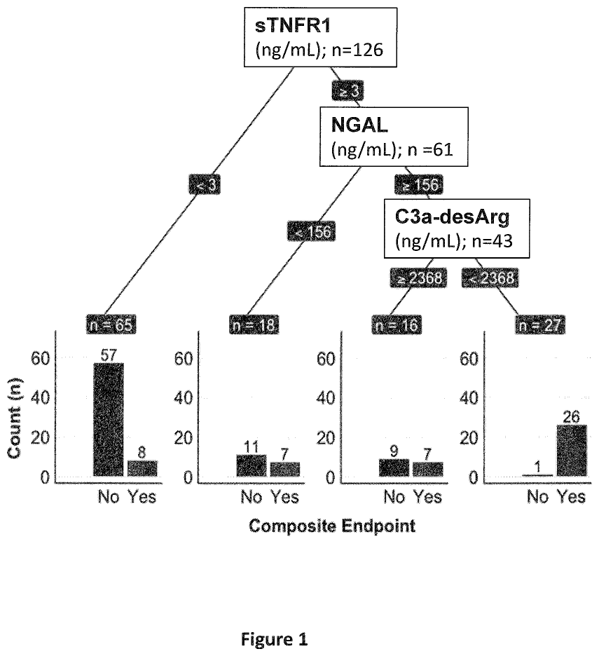

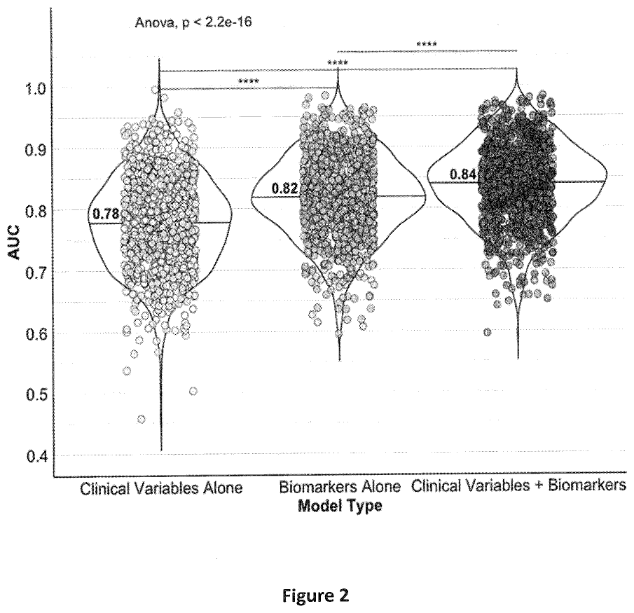

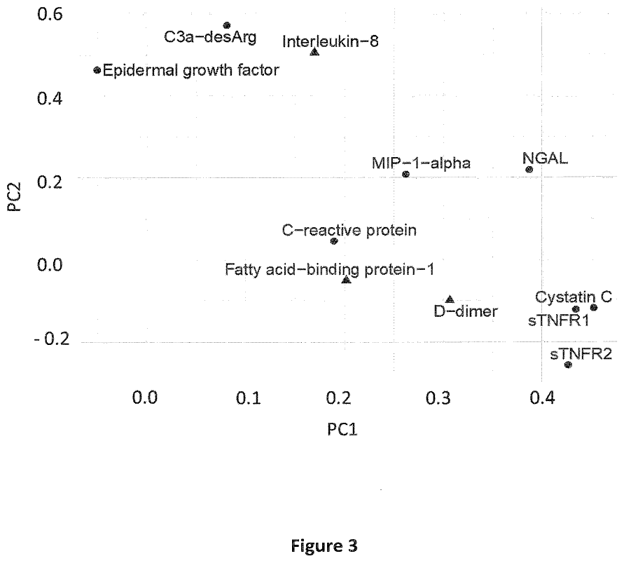

Alternatively, a method of the current invention can comprise determining the amount of sTNFR1 and NGAL in an ex vivo sample and establishing the significance of the level of the biomarkers by inputting each of the biomarker concentration values into a statistical methodology to produce an output value that indicates CKD prognosis for the patient. In the cohort of the current invention 77% of individuals with increased sTNFR1 and increased NGAL compared to reference values went on to develop the composite end-point at follow-up (FIG. 1). Either on its own or in combination with clinical factors this method would be useful in determining prognosis for individual CKD patients. The method can be improved further by the addition of C3a-desArg as described above. In the cohort of the current invention 96% of individuals with increased sTNFR1, increased NGAL and decreased C3a-desArg compared to reference values went on to develop the composite end-point at follow-up (FIG. 1).

The “level” of a biomarker refers to the amount, expression level or concentration of the biomarker within the sample. This level can be a relative level in comparison to another biomarker or a previous sample.

The term “adverse outcome” as used herein refers to a worsening or progression of CKD, specifically an adverse outcome is defined as a 40% decline in CKD-EPI eGFR, doubling of serum creatinine, need for renal replacement therapy, or death. The mean duration of renal functional follow-up was 4.1±1.6 years.

The term “patient” refers to any mammal to be the recipient of the diagnosis, preferably a human. Preferably the patients of the current invention are patients with previously diagnosed CKD. More preferably, the patients of the current invention are patients with Stage 3 CKD or greater. Stage 3 CKD may be further classified into stages 3a and 3b based on eGFR, with stage 3a having an eGFR of 45-59 and stage 3b having an eGFR of 30-44 mL/min/1.73 m². The patient may be a person presenting for a routine check-up or they may present with symptoms suggestive of worsening of their condition. The patient may also be an individual deemed at high risk for progression of CKD, due to comorbidities for example. Alternatively, the patient could be an individual who has received treatment for CKD and they are screened to monitor progress or detect possible progression of their condition.

The term “biomarker”, in the context of the current invention, refers to a molecule present in a biological sample of a patient, the levels of which may be indicative of progression of CKD. Such molecules may include peptides/proteins or nucleic acids and derivatives thereof.

The preferred biomarker combination of the current invention for determining the prognosis of CKD is sTNFR1, NGAL and C3a-desArg. However, it is within the scope of the invention to determine levels of additional biomarkers which could contribute to the determination of prognosis, for example, but not limited to IL-8, EGF, cystatin C, sTNFR2, D-dimer, FABP1, CRP and MIP-1 alpha.

The current invention provides a biomarker combination which allows high-risk CKD patients, i.e. those with a worse prognosis, to be identified. Monitoring of the progression of CKD is critical and identifying individuals with a worse prognosis can dramatically increase the patients' chances of survival. Additionally, the biomarker combination of the current invention allows the monitoring of CKD development within an individual through serial testing of samples from said individual over an extended period. For example, routine determination of the levels of the three biomarkers of the preferred combination could detect the changes from levels measured in previous samples from the individual, which can be indicative of the development of CKD. A further change in levels could then be indicative of the progression of the disease to a later stage. Such personalised testing can guide changes or intensification of treatment for the individual to improve their prognosis.

In the context of the present invention, a “reference or control value” is understood to be the level of a particular biomarker, such as sTNFR1, NGAL and C3a-desArg, typically found in healthy individuals or derived from individuals with CKD whose condition has not progressed within a specified timeframe. The control level of a biomarker may be determined by analysis of a sample isolated from a healthy individual or may be the level of the biomarker understood by the skilled person to be typical for a healthy individual. The reference value may be determined from a range of values considered by the skilled person to be a normal level for the biomarker in a healthy individual or a range of values of the biomarker found in individuals with CKD which has not progressed within a specified timeframe. The skilled person will appreciate that control values for a biomarker may be calculated by the user analysing the level of the biomarker in a sample from a healthy individual or by reference to typical values provided by the manufacturer of the assay used to determine the level of biomarker in the sample. The reference value may also be the level of a biomarker in a cohort which has been matched for age, gender or geographical location.

In the context of the present invention, a deviation from a control or reference value for a biomarker may be an indication that the patient has a worse prognosis and may require treatment intensification. Dependent on the individual biomarker this deviation may be an increase or a decrease from a control value. For example, in the patient cohort of the current invention, levels of C3a-desArg were lower in patients who developed an adverse renal outcome. Levels of sTNFR1 and NGAL were higher in patients who developed an adverse renal outcome.

In the study cohort of the current invention a C3a-desArg value of <2,368 ng/mL was indicative of an adverse outcome, an NGAL value of 156 ng/mL was indicative of an adverse outcome and an sTNFR1 value of 3 ng/mL was indicative of an adverse outcome (FIG. 1). Therefore, in one embodiment the reference value for sTNFR1 is about 3 ng/mL, the reference value for NGAL is about 156 ng/mL and the reference value for C3a-desArg is about 2,368 ng/mL. The skilled person will understand that these reference values are applicable to the current cohort and may change depending on the cohort or population of patients used. Reference values may also vary depending on the method which is used to measure the biomarker levels.

The “sample” of the current invention can be any ex vivo biological sample from which the levels of biomarkers can be determined. Preferably, the sample isolated from the patient is a whole blood, plasma or serum sample. Most preferably, the sample is a serum sample. The determination of the level of biomarkers may be carried out on one or more samples obtained from the patient. For example, one or more biomarkers could be measured in a serum sample and these results combined with those for one or more biomarkers which are measured in a urine sample from the same patient. The sample may be obtained from the patient by methods routinely used in the art.

The determination of the level of biomarkers in the sample may be determined by immunological methods such as an ELISA-based assay. The methods of the current invention preferably comprise the following steps; the biomarkers binding to a probe(s), adding a detector probe(s) and detecting and measuring the biomarker/probe complex signal(s), placing these values into a machine algorithm and analysing the output value, said value indicating the patient's prognosis. Preferably, the methods of the present invention use a solid-state device for determining the level of biomarkers in the sample isolated from the patient.

The solid-state device comprises a substrate having a probe or multiple different probes immobilised upon it that bind specifically to a biomarker. The interactions between a biomarker and its respective probe can be monitored and quantified using various techniques that are well-known in the art. The term “probe” refers to a molecule that is capable of specifically binding to a target molecule such that the target molecule can be detected as a consequence of said specific binding. Probes that can be used in the present invention include, for example, antibodies, aptamers, phages and oligonucleotides. In a preferred embodiment of the current invention the probe is an antibody.

The term “antibody” refers to an immunoglobulin which specifically recognises an epitope on a target as determined by the binding characteristics of the immunoglobulin variable domains of the heavy and light chains (VHS and VLS), more specifically the complementarity-determining regions (CDRs). Many potential antibody forms are known in the art, which may include, but are not limited to, a plurality of intact monoclonal antibodies or polyclonal mixtures comprising intact monoclonal antibodies, antibody fragments (for example Fab, Fab′, and Fv fragments, linear antibodies single chain antibodies and multispecific antibodies comprising antibody fragments), single-chain variable fragments (scFvs), multi-specific antibodies, chimeric antibodies, humanised antibodies and fusion proteins comprising the domains necessary for the recognition of a given epitope on a target. Preferably, references to antibodies in the context of the present invention refer to polyclonal or monoclonal antibodies. Antibodies may also be conjugated to various reporter moieties for a diagnostic effect, including but not limited to radionuclides, fluorophores, dyes or enzymes including, for example, horse-radish peroxidase and alkaline phosphatase.

Such antibodies may be immobilised at discrete areas of an activated surface of the substrate. The solid-state device may perform multi-analyte assays such that the level of a biomarker in a sample isolated from the patient may be determined simultaneously with the level of a further biomarker of interest in the sample. In this embodiment, the solid-state device has a multiplicity of discrete reaction sites each bearing a desired antibody covalently bound to the substrate, and in which the surface of the substrate between the reaction sites is inert with respect to the target biomarker. The solid-state, multi-analyte device may therefore exhibit little or no non-specific binding. The combination of biomarkers may also be referred to as a panel of biomarkers.

The substrate can be any surface able to support one or more probes but is preferably a biochip. A biochip is a planar substrate that may be, for example, mineral or polymer based, but is preferably ceramic. When identifying the various biomarkers/proteins of the invention it will be apparent to the skilled person that as well as identifying the full-length protein, the identification of a fragment or several fragments of a protein is possible, provided this allows accurate identification of the protein. Similarly, although a preferred probe of the invention is a polyclonal or monoclonal antibody, other probes such as aptamers, molecular imprinted polymers, phages, short chain antibody fragments and other antibody-based probes may be used.

A solid-state device that may be used in the invention may be prepared by activating the surface of a suitable substrate and applying an array of antibodies on to the discrete sites on the surface. If desired, the other active areas may be blocked. The ligands may be bound to the substrate via a linker. In particular, it is preferred that the activated surface is reacted successively with an organosilane, a bi-functional linker and the antibody. The solid-state device used in the methods of the present invention may be manufactured according to the method disclosed in, for example, GB-A-2324866 the contents of which are incorporated herein in its entirety. The solid-state device can be any substrate to which probes of the current invention can be attached for example a microtitre plate or beads. Preferably, the solid-state device used in the methods of the present invention is a biochip. The biochip may be a biochip which is incorporated into the Biochip Array Technology System (BAT) available from Randox Laboratories Limited (Crumlin, UK).

Preferably, a solid-state device may be used to determine the levels of sTNFR1, NGAL and C3a-desArg in the sample isolated from the patient. In a preferred embodiment the solid-state device comprises a substrate having an activated surface on to which is applied antibodies specific to each of the two or more biomarkers to discrete areas of the activated surface. Therefore, the solid-state device may perform multi-analyte assays such that the levels of biomarkers, for example sTNFR1, NGAL and C3a-desArg in a sample may be determined simultaneously. In this embodiment, the solid-state device has a multiplicity of discrete reaction sites each bearing a desired antibody covalently bound to the substrate, and in which the surface of the substrate between the reaction sites is inert with respect to the target biomarkers. Each probe, whether individually or in multiplex, is specific to one target analyte. For example, a probe to sTNFR1 will only show specific binding to this analyte and will have no significant cross-reactivity with NGAL, C3a-desArg or indeed any other potentially interfering substance which could compromise the assay. When two different sample types are obtained from a patient the solid-state device of the invention can consist of two identical solid-state devices with the same antibodies to the same biomarkers or it may consist of two separate solid-state devices, one for each sample type, comprising the antibodies specific to the biomarkers which are to be determined in each sample type. Conceivably, the solid-state device could be three separate devices each comprising antibodies specific to a different target biomarker. Or, for example, the solid-state device could be one device with probes to sTNFR1, NGAL and C3a-desArg or it could be two separate devices, one with probes to sTNFR1 and another with probes to NGAL and C3a-desArg. The solid-state device not only has potential in prognosis of CKD but also in monitoring the progression and determining the success of treatments.

In a preferred embodiment of the current invention each of the biomarker concentration values is inputted into a statistical methodology to produce an output value that correlates with the patients CKD prognosis. Preferably, the statistical methodology used is logistic regression, decision trees, support vector machines, neural networks, random forest or another machine-learning algorithm.

A suitable statistical classification model, such as logistic regression, can be derived for a combination of biomarkers. Moreover, the logistic regression equation can be extended to include other (clinical) variables such as age and gender of the patient as well. In the same manner as described before, the ROC curve can be used to access the performance of the discrimination between patients and controls by the logistic regression model. Therefore, the logistic regression equation can be used apart or combined with other clinical characteristics to aid clinical decision making. Although a logistic regression equation is a common statistical procedure used in such cases and is preferred in the context of the current invention, other mathematical/statistical, decision trees or machine learning procedures can also be used.

One convenient goal to quantify the diagnostic accuracy of a laboratory test is to express its performance by a single number. The most common global measure is the area under the curve (AUC) of the ROC plot. The area under the ROC curve is a measure of the probability that the perceived measurement will allow correct identification of a condition. Values typically range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). In the context of the present invention, the two different conditions can be whether a patient is high risk for a worse prognosis or not.

A further aspect of the present invention is a method of determining the efficacy of a treatment for CKD comprising determining the levels of sTNFR1, NGAL and C3a-desArg in a sample from a patient who has had treatment for CKD and, comparing levels with those from a reference value, a healthy control or with levels from the same patient taken before the treatment, wherein dependent on the biomarker, either an increase or decrease in level indicates the effectiveness of the treatment. The treatment can be for example, lifestyle changes, a drug treatment, a dialysis-based treatment or a surgical intervention (e.g. transplantation). Wherein the treatment is a drug treatment, the method of determining the efficacy of the drug treatment for CKD would comprise determining the levels of biomarkers, for example sTNFR1, NGAL and C3a-desArg in a sample from a patient treated with the drug, and comparing biomarker levels with those from a healthy control or with levels from the same patient before treatment with the drug, wherein, dependent on the biomarker, either an increase or decrease in level indicates the effectiveness of the drug treatment.

Methods

Study Cohort

Adults with CKD stages 1 to 5 were enrolled from nephrology outpatient clinics at Galway University Hospitals between February 2014 and November 2016. As previously described (8), inclusion criteria for the study cohort were: (a) Age years, (b) Diagnosis of CKD based on eGFR, urinalysis and/or renal imaging, (c) Not currently being treated for infection, cancer, acute cardiovascular event or haematological condition other than anaemia, (d) Willing and able to provide informed consent, (e) Haemoglobin level ≥10 g/dL, (f) Not currently receiving chronic haemodialysis or peritoneal dialysis, (g) Not the recipient of a kidney transplant, (h) Not known to be positive for human immunodeficiency virus, hepatitis B virus or hepatitis C virus, (i) Not currently receiving immunosuppressive therapy.

Clinical and laboratory data were recorded from study enrolment to the end of study follow-up on Jul. 15, 2020 in a secure, password-protected, web-based clinical database (Distiller®, SlidePath, Dublin, Ireland). Electronic medical records of the enrolled study subjects were reviewed by medically-qualified members of the research team and relevant fields were compiled in the database. Determination of mortality, renal (≥40% decline in Chronic Kidney Disease-Epidemiology Collaboration (CKD-EPI) eGFR, doubling of serum creatinine, need for renal replacement therapy), and composite endpoints was performed manually for each patient on a case-by-case basis. Time to event was recorded for each relevant endpoint reached.

Laboratory Data

Longitudinal measurements of serum creatinine concentration were extracted for each participant using the eMEDRenal clinical data system (Mediqal H. I., Aston, UK) from the date of enrolment to the end of study follow-up on Jul. 15, 2020. An IDMS-traceable creatininase assay was used to measure creatinine at the Galway University Hospitals Clinical Biochemistry laboratory. CKD-EPI eGFR was calculated from serum creatinine using standard formulae and expressed as mL/min/body surface area (BSA). Only patients with eGFR determinations over year were included in analyses of longitudinal renal functional data. Second and subsequent creatinine values on a given day and serum creatinine values subsequent to renal replacement therapy initiation were excluded. Annual slopes of CKD-EPI eGFR were calculated by linear regression of eGFR over time in years.

Blood Sample Collection and Processing

Blood sample collection was performed once for each participant at the time of consent. Freshly-drawn peripheral venous blood samples were collected into serum separator tubes and allowed to coagulate for 2 hours at room temperature in a horizontal position. The tubes were then centrifuged at 800 RCF for 15 minutes at room temperature with full acceleration and brake setting 2. The supernatant (serum) was carefully removed from each tube using a 3 mL Pasteur pipette and aliquoted into 2 mL screw-top Eppendorf tubes which were stored at −80° C. until use.

Serum Biomarker Determinations

Quantification of 11 serum biomarkers was performed using two CKD multiplex arrays utilising an Evidence Investigator® immunoassay analyser (Randox Teoranta, Co. Donegal, Ireland). The first 7-analyte array measured biomarkers of lower abundance including epidermal growth factor (EGF), interleukin-8, soluble tumour necrosis factor receptor-1 (sTNFR1), soluble tumour necrosis factor receptor-2 (sTNFR2), fatty acid-binding protein-1 (FABP1), D-dimer, macrophage inflammatory protein-1-alpha (MIP-1-alpha); the second 4-analyte array measured biomarkers of higher abundance including C-reactive protein, cystatin C, complement protein C3a with cleaved C-terminal arginine (C3a desArg), and neutrophil gelatinase-associated lipocalin (NGAL).

Calibrator standards and controls for both arrays were re□suspended in 1 mL of deionized water. Controls and calibrators were rolled for 30 minutes at room temperature. The biochips were prepared by first adding 200 μL assay buffer to each well and then 100 μL of either calibrators, controls or samples. The biochips were incubated for 1 hour at 37° C. on a gentle shaker set to 370 RPM. The supernatant was removed using a gentle flicking motion and washed twice with wash buffer, with a further 4 wash cycles completed allowing a 2-minute incubation between each wash. The biochips were dried by gently tapping them on lint-free paper and 300 μL of specific conjugate was added to each well. The biochips were incubated for 1 hour at 37° C. on a gentle shaker set to 370 RPM. Supernatant was removed, and the wash steps were carried out as described above. The biochips were dried by gently tapping them on lint-free paper and 250 μL chemiluminescent solution was added to each well. The biochips were incubated for 2 minutes away from light prior to analysis using the Evidence Investigator® analyser (Randox Teoranta, Co. Donegal, Ireland). For the first 7-analyte array (lower abundance proteins), serum samples were undiluted. For the second 4-anlayte array, serum samples were diluted at 1:200 in sample diluent. Each day, the first carrier from each kit was used for calibration (9 wells) and the second carrier was used for controls (3 controls in duplicate). Samples were tested in duplicate.

Statistical Analyses

RStudio® version 4.0.0 was used for analysis. A composite renal and mortality endpoint consisting of 40°/o decline in CKD-EPI eGFR, doubling of serum creatinine, need for renal replacement therapy, or death was defined as the primary outcome of interest. Baseline characteristics at study enrolment and clinical characteristics of the cohort during follow-up (renal and mortality endpoints) were summarised by descriptive statistics. Categorical variables are presented as frequencies and percentages and were compared between those who did and did not develop the composite endpoint using χ² tests. Continuous variables with normal and skewed distributions are presented as mean±SD and median [interquartile range], respectively. Independent sample t-tests and Wilcoxon rank-sum tests were used to assess for differences amongst those who did and did not develop the composite endpoint in continuous variables with normal and skewed distributions, respectively. P<0.05 was considered statistically significant.

Serum biomarkers were log-transformed for analysis due to non-Gaussian distributions. Hierarchial clustering was performed to examine expression patterns of the 11 serum biomarkers; results are presented visually on a heatmap generated with the R package pheatmap (9). Unsupervised clustering of biomarkers by principal components analysis was performed, with results presented visually (R package factoextra) to assess for shifts in biomarker expression by CKD stage and by development of the composite endpoint (10). Univariate relationships between baseline eGFR, serum biomarker concentrations and the composite endpoint were investigated using logistic regression.

Due to collinearity amongst biomarkers affecting predictions from logistic regression models, a supervised machine learning approach with binary classification random forests was used to explore the value of all biomarkers considered together in predicting the composite endpoint. Firstly, binary classification trees were generated and plotted using the R packages rpart and ggparty, respectively, to illustrate the complementary information provided by multiple biomarkers (11, 12). Binary classification random forests, with the composite endpoint as the response variable, were fit using the R package randomForest (13). Separate models were created for clinical variables alone (age, gender, diabetes, and baseline eGFR), serum biomarkers alone, and clinical variables plus biomarkers; 5,000 trees were grown per model. A training set consisting of 75% of the study cohort was randomly sampled, upon which the random forest model was trained. The model was subsequently tested on the remaining 25% of the cohort, which served as the previously unseen test set, and an area under the curve (AUC) value for classification of the composite endpoint by the model was calculated to quantify predictive performance of the model. This process was repeated 1,000 times for each of the three model types, with repeated random sampling of training and test sets in each iteration. Mean AUC values from 1,000 iterations were compared for the 3 model types by ANOVA, with post-hoc testing was performed by multiplicity-corrected independent samples t-tests. Variable importance estimates presented were calculated from mean values of 1,000 random forest model iterations for each of the 3 model types, this time using all of the study cohort to maximise the amount of data provided to train the models.

Multivariable Cox proportional hazards models were created to further investigate relationships between serum biomarkers and time to the composite endpoint. Two models were constructed for each endpoint: firstly, a clinical model adjusting for conventional risk factors for renal functional decline (age, gender, diabetes status, and CKD-EPI eGFR); and secondly a clinical+serum biomarker model incorporating the aforementioned variables and serum biomarkers. Backward elimination of non-significant effects from the clinical+biomarker model was performed using the function stepAlC from the R package MASS (14); the aforementioned clinical variables were manually retained in the parsimonious model. Cox models were constructed using the R package survival (15); adjusted survival curves were plotted using the R package survminer according to biomarker tertiles (16). We tested each Cox proportional hazards model for proportionality assumptions using Schoenfeld residuals. Cox model results are presented in Forest plots with the hazard ratio (HR), 95% confidence interval (CI), and p-value displayed. Comparisons of model adequacy (clinical model versus clinical+biomarker model) were assessed for linear mixed-effects and Cox models using likelihood ratio X² tests.

Linear mixed-effects models were constructed to investigate relationships between serum biomarkers and rate of change of CKD-EPI eGFR. Separate models were fitted for CKD-EPI eGFR and log-transformed CKD-EPI eGFR. A clinical model was constructed that adjusted for age, gender, diabetes status, and CKD-EPI eGFR; a clinical+biomarker model was created which additionally adjusted for serum biomarkers. sTNFR2 was omitted from the clinical+biomarker model due to collinearity with sTNFR1 resulting in paradoxical and unstable model predictions. Backward elimination of non-significant effects from the clinical +biomarker model was performed using the function step from the R package ImerTest (17). All linear mixed-effects models incorporated subject-specific random intercepts and slopes for the duration of renal functional follow-up. Models fitted to the absolute eGFR values determine changes in renal function during study follow-up in native units (mL/min/BSA), whereas models fitted using log-transformed eGFR estimate percentage changes in renal function from baseline. The function Imer (from the R package ImerTest) was used to fit and test the models (17). No serious violations of linear mixed-effects models were found on examination of the distribution of residuals. Coefficients of model fixed-effects were plotted using the dotwhisker R package (18).

To compensate for missing baseline uACR data for the study cohort, median log-transformed uACR was estimated from uPCR values in those with available data using a validated equation recently developed by Weaver et al. (19). Sensitivity analyses were performed in which baseline log-transformed uACR (directly measured or calculated from uPCR) was included as an additional clinical covariate in Cox proportional hazards regression and linear mixed-effects models.

Results

Baseline Characteristics and Serum Biomarker Concentrations

Table 1 presents baseline characteristics and serum biomarker concentrations of the study cohort (n=139), stratified by development (n=56) or not (n=83) of the composite endpoint. As shown, the study population had a mean age of 63 years, 56% were male, and median [IQR] CKD-EPI eGFR was 33 [26.5] mL/min/BSA). Most patients had CKD stage 3 or greater at enrolment; just over 15% had CKD stages 1 or 2, while over 45% and 35% were classified as having CKD stage 3 and CKD stages 4 or 5, respectively. Baseline uACR and uPCR data were missing for 58% and 45% of the study cohort, respectively. Of those sampled, study participants had moderate proteinuria with a median [IQR] uACR of 14 mg/mmol and uPCR of 37 mg/mmol. After calculation of uACR from uPCR using the validated equation of Weaver et al. (19), 81% of the study cohort had baseline uACR data available. Median [IQR] uACR for these 113 participants was 16.3 [57.6] mg/mmol. Over 25% and 80% of the study cohort had diabetes mellitus and hypertension, respectively, while glomerulonephritis (22%) and diabetic kidney disease (17%) were the two most documented CKD aetiologies.

Compared with individuals who did not develop the composite endpoint during follow-up, those who did were older (67±15 vs 60±17 years, p=0.01), more likely to be male (71% vs 46%, p=0.005), had a higher prevalence of diabetes mellitus (43% vs 13%, p<0.001), lower eGFR at enrolment (26 vs 43 mL/min/BSA, p<0.001), and higher uACR (32 vs 12 mg/mmol, p<0.001). Concentrations of several serum biomarkers from the multi-analyte assay were higher in those who developed the composite endpoint, including sTNFR1, sTNFR2, NGAL, cystatin C, and to a lesser extent C-reactive protein, FABP1, and MIP-1-alpha. Conversely, concentrations of EGF and C3a-desArg were lower in those who developed the composite endpoint. Interleukin-8 and D-dimer concentrations were not significantly different between the two groups.

Incidence of Renal and Mortality Endpoints

Mean duration of renal functional follow-up was 4.1±1.6 years, with participants having a median of 22 eGFR determinations (Table 2). The median rate of annual decline in CKD-EPI eGFR was −0.9 [3.1] mL/min/BSA/year for the study cohort. Annual decline in kidney function was greater in those who developed the composite endpoint compared with those who did not (−2.4 [3.1] vs 0 [2.7] mL/min/BSA, p<0.001). Individual components of the composite renal and mortality endpoint are also presented in Table 2: of the 56 individuals who reached this endpoint during follow-up, 38 (68%), 14 (25%), 21 (38%), and 15 (27%) reached it for ≥40% decline in CKD-EPI eGFR, doubling of serum creatinine, new requirement for renal replacement therapy, and death, respectively.

Univariate Relationships Between Serum Biomarkers at Enrolment and a Composite Renal and Mortality Endpoint

Biomarkers which exhibited statistically significant (p<0.05) associations with development of the composite renal and mortality endpoint by univariate logistic regression were EGF, C3a-desArg, MIP-1-alpha, CRP, STNFR1, sTNFR2, NGAL and Cystatin C. Interleukin-8, D-dimer, and FABP1 were not significantly associated with development of the composite endpoint. Both EGF (p=0.02) and C3a-desArg (p=0.04) were inversely associated with, while both MIP-1-alpha (p=0.005) and C-reactive protein (p=0.002) were positively associated with, development of the composite outcome. Furthermore, soluble TNF receptors 1 and 2, NGAL, and cystatin C were had strong positive associations with development of the composite endpoint (all p<0.001). Those with adverse serum biomarker signatures were also more likely to have advanced CKD.

Hierarchial Clustering and Principal Components Analysis Illustrate Relationships between Biomarker Expression, CKD Stage, and a Composite Renal and Mortality Endpoint

Expression of several serum biomarkers clustered together across the study cohort in a predictable fashion. For example, levels of soluble TNF receptors 1 and 2 were very similar to each other. The chemokines interleukin-8 and MIP-1-alpha also clustered together, as did two biomarkers which were inversely associated with development of the composite endpoint: EGF and C3a-desArg. Thus, clustering of biomarkers in a fashion which is expected based on a priori knowledge provides validation of the technical accuracy of the Biochip platform. Those with adverse biomarker signatures were more likely to have advanced CKD and more frequently developed the composite endpoint.

Unsupervised clustering of biomarkers by principal components analysis identified global shifts in serum biomarker profiles by CKD stage and by development of the composite endpoint. The principal components loading plot (FIG. 3) provides insight into which biomarkers separated patients by CKD stage and by the composite endpoint. Biomarkers clustered in the upper left corner of the plot (interleukin-8, EGF, and C3a-desArg) were more strongly expressed in earlier stage CKD and in those who did not develop the composite endpoint—that is to say they were inversely associated with the composite endpoint. Conversely, biomarkers clustering to the far right of the loadings plot (NGAL, cystatin C, sTNFR1, and sTNFR2) were more strongly expressed in advanced CKD stages and in those who developed the composite endpoint. Biomarkers in the middle of the loadings plot (D-dimer, FABP1, C-reactive protein, and MIP-1-alpha) had less discriminant value in separating the study cohort by CKD stage or by the composite endpoint.

Complementary Prognostication Provided by Serum Biomarkers Improves Prediction of a Composite Renal and Mortality Endpoint Over Clinical Variables Alone (Random Forest Classification Models)

FIG. 1 presents binary classification of the composite endpoint by serum biomarkers in the study cohort. Individuals with low sTNFR1 values (<3 ng/mL) had a relatively low risk of the composite endpoint (12.3%). However, not all individuals with high sTNFR1 values (≥3 ng/mL) had the same risk of the composite endpoint: those with low NGAL values (<156 ng/mL) or high NGAL values (≥156 ng/mL) coupled with high C3a-desArg values (≥2,368 ng/mL) had risks of the composite endpoint in the order of 38.9% and 43.8%, respectively. Conversely, individuals with the triad of high sTNFR1 (≥3 ng/mL), high NGAL (≥156 ng/mL), and low C3a-desArg (<2,368 ng/mL), which accounted for almost 20% of the study cohort, almost universally (96.3%) developed the composite endpoint during follow-up.

AUC values calculated from binary classification of the composite endpoint by random forests with repetitive random sampling of training (75%) and test (25%) sets over 1,000 iterations are presented in FIG. 2. An incremental improvement in predictive performance, represented by mean AUC values, was observed between random forests trained on clinical variables alone and on serum biomarkers alone (AUC 0.78 vs 0.82, p<0.001). Furthermore, predictive performance of biomarkers was enhanced after inclusion of clinical variables alongside biomarkers to train the random forest models (AUC 0.84 vs 0.82 for biomarkers alone, p<0.001). Variable importance to correct prediction of the composite endpoint by trained random forest models was ranked to provide insight into relative importance of clinical and biomarker covariates. Baseline eGFR was by far the most important predictor of the composite endpoint in models trained on clinical variables alone, while when incorporated alongside serum biomarkers, eGFR and cystatin C were ranked as the third and fourth most important variables to classification by the models, respectively. Serum sTNFR1 and NGAL were found to be the top 2 most important variables for correct prediction of the composite endpoint, both when biomarkers were considered alone and alongside clinical variables, highlighting the strength of the associations between these parameters and adverse renal and mortality outcomes which in fact superseded that of clinical covariates in our study cohort.

C-Reactive Protein, NGAL, and C3a-desArg Independently Predict Time to Onset of a Composite Renal and Mortality Endpoint (Cox Proportional Hazards Regression Models)

A Forest plot of a parsimonious multivariate Cox proportional hazards regression model was determined for the composite endpoint, incorporating clinical variables and serum biomarkers which improved model performance. Compared with a Cox model incorporating the same clinical variables, the clinical plus biomarker model displayed significantly improved prediction of time to the composite endpoint (p=0.001 by likelihood ratio test). C-reactive protein (HR 1.4, p=0.02) and NGAL (HR 2.7, p=0.01) were positively associated with time to the composite endpoint, with an almost 3-fold elevation in risk of the composite endpoint per logarithm increase in baseline serum NGAL. A similar trend was observed for sTNFR1, which did not reach statistical significance (HR 2.4, p=0.07). C3a-desArg values were inversely associated with time to the composite endpoint (HR 0.5, p=0.02), with a halving of risk in the composite endpoint per logarithm increase in baseline C3a-desArg.

A limitation to these analyses was the lack of a uniform baseline quantification of albuminuria/proteinuria. To test whether the added predictive value of uACR may negate that observed for some of the serum biomarkers, baseline log-transformed uACR (either directly measured or calculated from uPCR using the equation of Weaver et al. (19)) was incorporated into Cox models as an additional clinical covariate. Compared with a Cox model of clinical variables alone, incorporation of serum biomarkers again improved prediction of time to the composite endpoint (p=0.003). As expected, baseline uACR independently predicted time to the composite endpoint (HR 1.3, p=0.03). C-reactive protein (HR 1.5, p=0.01) and NGAL (HR 3.5, p=0.004) remained predictive of time to the composite endpoint, with effect sizes of a similar magnitude to those obtained prior to adjustment for baseline uACR. However, a positive trend between sTNFR1 and the composite endpoint was no longer observed (HR 1.04, p=0.95). An inverse trend between C3a-desArg and the composite endpoint persisted, but was no longer statistically significant (HR 0.6, p=0.07).

Serum NGAL Independently Predicts Changes in eGFR Slope Trajectory During Follow-Up (Linear Mixed-Effects Models)

Compared with clinical variables alone, incorporation of serum biomarkers alongside clinical variables improved prediction of absolute (p=0.004) and percentage (p<0.001) changes in CKD-EPI eGFR during study follow-up. Serum NGAL predicted a more negative eGFR trajectory during follow-up: −3.8 [95% CI −1.6-−5.9] mL/min/BSA, p<0.001 and −17.8 [95% CI −9.7-−25.8] % loss of baseline renal function, p<0.001 per unit increase in baseline log NGAL. C3a-desArg predicted a more positive eGFR trajectory: +7.9 [95% CI 2.5-13.3] change from baseline eGFR, p=0.005 per unit increase in baseline log C3a-desArg. A similar trend was present for absolute changes in renal function (+1.4 [95% CI −0.1-2.9] mL/min/BSA, p=0.06 per unit increase in log C3a-desArg), but did not reach statistical significance. FABP1 (−5.3 [95% CI −0.5-−10.0] % per log unit increase, p=0.03) and sTNFR1 (−11.1 [95% CI −1.0-−21.3] % per log unit increase, p=0.03) also predicted percentage losses in renal function.

In a sensitivity analysis, log-transformed uACR was incorporated into linear mixed-effects models as an additional clinical covariate. Compared with linear mixed-effects models of clinical variables alone, incorporation of serum biomarkers again improved prediction of absolute (p=0.04) and percentage (p<0.001) changes in eGFR. Serum NGAL remained predictive of a more negative eGFR trajectory during follow-up: −3.3 [95% CI −0.8-−5.9] mL/min/BSA, p=0.01 and −17.5 [95% CI −8.8-−26.2] % loss of baseline renal function, p <0.001 per unit increase in log NGAL). C3a-desArg remained predictive of a more positive eGFR trajectory (+6.9 [95% CI 1.3-12.6] % change from baseline eGFR, p=0.02 per unit increase in log C3a-desArg). FABP1 (−5.4 [95% CI −0.4-−10.4] % renal functional loss per log unit increase, p=0.04) also remained predictive of a more negative trajectory in percentage eGFR, but sTNFR1 (−11.1 [95% CI 2.5-−24.7] % renal functional loss per log unit increase, p=0.11) was no longer predictive after adjustment for baseline uACR.

TABLE 1
Baseline Characteristics and Serum Biomarker Concentrations of the Study Cohort
Stratified by Development of the Composite Endpoint (n = 139).^{a, b, c}

| | | | | | Did not | | | |
|---|---|---|---|---|---|---|---|---|
| | | | | | develop | | Developed | |
| | Data | | Total | | composite | | composite | |
| | available | | cohort | | endpoint | | endpoint | |
| Characteristic | (n (%)) | | (n = 139) | | (n = 83) | | (n = 56) | |
| Clinical parameters | | | | | | | | |
| Age (mean ± SD; years) | 139 | (100) | 63 ± 17 | | 60 ± 17 | | 67 ± 15 | |
| Male (n (%)) | 139 | (100) | 78 | (56) | 38 | (46) | 40 | (71) |
| Diabetes mellitus (n (%)) | 139 | (100) | 35 | (25) | 11 | (13) | 24 | (43) |
| Hypertension (n (%)) | 137 | (99) | 113 | (83) | 64 | (77) | 49 | (91) |
| Coronary artery disease | 139 | (100) | 18 | (13) | 8 | (10) | 10 | (18) |
| (n (%)) | | | | | | | | |
| CKD stage (n (%)) | 139 | (100) | | | | | | |
| Grade 1 | | | 5 | (4) | 5 | (6) | 0 | (0) |
| Grade 2 | | | 17 | (12) | 16 | (19) | 1 | (2) |
| Grade 3a | | | 22 | (16) | 19 | (23) | 3 | (5) |
| Grade 3b | | | 42 | (30) | 25 | (30) | 17 | (30) |
| Grade 4 | | | 46 | (33) | 16 | (19) | 30 | (54) |
| Grade 5 | | | 7 | (5) | 2 | (2) | 5 | (9) |
| CKD aetiology (n (%)) | 139 | (100) | | | | | | |
| Diabetes | | | 23 | (17) | 8 | (10) | 15 | (27) |
| Hypertension | | | 12 | (9) | 9 | (11) | 3 | (5) |
| Glomerulonephritis | | | 30 | (22) | 23 | (28) | 7 | (13) |
| Congenital | | | 8 | (6) | 7 | (8) | 1 | (2) |
| Polycystic kidney disease | | | 6 | (4) | 3 | (4) | 3 | (5) |
| Obstructive | | | 6 | (4) | 2 | (2) | 4 | (7) |
| Interstitial | | | 7 | (5) | 5 | (6) | 2 | (4) |
| Other/unknown | | | 47 | (34) | 26 | (31) | 21 | (38) |
| Laboratory data | | | | | | | | |
| Serum creatinine | 139 | (100) | 2.0 ± 1.0 | | 1.6 ± 0.6 | | 2.6 ± 1.1 | |
| (mean ± SD; mg/dL) | | | | | | | | |
| CKD-EPI eGFR (median | 139 | (100) | 33 | [27] | 43 | [29] | 26 | [16] |
| [IQR]; mL/min/BSA) | | | | | | | | |
| uACR (median [IQR]; mg/g) | 58 | (42) | 127 | [457] | 33 | [126] | 504 | [1158] |
| uPCR (median [IQR]; mg/g) | 76 | (55) | 327 | [690] | 301 | [513] | 345 | [1199] |
| Merged uACR (median | 113 | (81) | 144 | [510] | 104 | [250] | 283 | [910] |
| [IQR]; mg/g)* | | | | | | | | |
| Haemoglobin (mean+SD; | 138 | (99) | 13.0 ± 1.7 | | 13.4 ± 1.9 | | 12.3 ± 1.2 | |
| g/dL) | | | | | | | | |
| Multiplex biomarker values | | | | | | | | |
| C-reactive protein (median | 139 | (100) | 3035 | [6637] | 2610 | [3881] | 4612 | [12878] |
| [IQR]; ng/ml) | | | | | | | | |
| Cystatin C (median [IQR]; | 139 | (100) | 3715 | [2425] | 3241 | [2269] | 4984 | [2077] |
| ng/ml) | | | | | | | | |
| D-dimer (median [IQR]; | 138 | (99) | 81 | [112] | 73 | [110] | 103 | [123] |
| ng/mL) | | | | | | | | |
| C3a-desArg (median [IQR]; | 139 | (100) | 1762 | [1867] | 2240 | [2293] | 1467 | [1230] |
| ng/ml) | | | | | | | | |
| EGF (median [IQR]; pg/mL) | 138 | (99) | 91 | [72] | 109 | [76] | 72 | [57] |
| FABP1 (median [IQR]; | 139 | (100) | 1.6 | [2.2] | 1.3 | [2.1] | 1.9 | [2.7] |
| ng/ml) | | | | | | | | |
| Interleukin-8 (median [IQR]; | 139 | (100) | 3.7 | [5.9] | 3.9 | [6.1] | 3.6 | [4.6] |
| pg/mL) | | | | | | | | |
| MIP-1-alpha (median [IQR]; | 130 | (94) | 4.8 | [8.0] | 3.9 | [4.3] | 7.5 | [11.4] |
| pg/mL) | | | | | | | | |
| NGAL (median [IQR]; ng/ml) | 137 | (99) | 167 | [137] | 137 | [90] | 214 | [155] |
| STNFR1 (median [IQR]; | 136 | (98) | 3.0 | [2.5] | 2.4 | [1.3] | 4.6 | [3.3] |
| ng/ml) | | | | | | | | |
| STNFR2 (median [IQR]; | 138 | (99) | 1.4 | [1.4] | 1.1 | [0.9] | 2.0 | [1.2] |

ng/ml)

^{a}BSA = body surface area; CKD-EPI = Chronic Kidney Disease-Epidemiology Collaboration; C3a-desArg = complement protein C3a (cleaved at C-terminal arginine); EGF = epidermal growth factor; eGFR = estimated glomerular filtration rate; FABP1 = fatty acid-binding protein-1; IQR = interquartile range; MIP-1-alpha = macrophage inflammatory protein-1-alpha; NGAL = neutrophil gelatinase-associated lipocalin; SD = standard deviation; STNFR1 = soluble tumour necrosis factor receptor-1; sTNFR2 = soluble tumour necrosis factor receptor-2; uACR = urine albumin-to-creatinine ratio; uPCR = urine protein-to-creatinine ratio
^{b}Values are given as n (%) for categorical variables, or mean ± SD for normally distributed continuous variables, unless otherwise indicated. Median [IQR] values are presented for continuous variables that are not normally distributed.
^{c}Composite endpoint: ≥40% decrease in CKD-EPI eGFR, doubling of serum creatinine, renal replacement therapy, or mortality.
^{d}Merged uACR represents a combination of measured uACR and calculated uACR from uPCR using the validated equation of Weaver et al. uACR values on the natural log scale were exponentiated such that presented values are in absolute units in mg/g.

TABLE 2
Duration of Renal Functional Follow-up and Incidence of Renal and
Mortality Endpoints During the Study Period (n = 139).^{a, b, c}

| | | | | | Did not | | | |
|---|---|---|---|---|---|---|---|---|
| | | | | | develop | | Developed | |
| | Data | | Total | | composite | | composite | |
| | available | | cohort | | endpoint | | endpoint | |
| Characteristic | (n (%)) | | (n = 139) | | (n = 83) | | (n = 56) | |
| Total number of eGFR | 139 | (100) | 22 | [22] | 19 | [18] | 27 | [26] |
| measurements (median | | | | | | | | |
| [IQR]) | | | | | | | | |
| Total duration of renal | 139 | (100) | 4.1 ± 1.6 | | 4.5 ± 1.1 | | 3.5 ± 1.9 | |
| functional follow-up | | | | | | | | |
| (mean+SD; years) | | | | | | | | |
| Slope of CKD-EPI eGFR | 129 | (93) | −0.9 | [2.8] | 0 | [2.6] | −2.2 | [2.5] |
| (median [IQR]; | | | | | | | | |
| mL/min/BSA/year)^{d} | | | | | | | | |
| >40% decrease in CKD-EPI | 139 | (100) | 38 | (27) | 0 | (0) | 38 | (68) |
| eGFR (n (%)) | | | | | | | | |
| Doubling of serum creatinine | 139 | (100) | 14 | (10) | 0 | (0) | 14 | (25) |
| (n (%)) | | | | | | | | |
| Required RRT (n (%)) | 139 | (100) | 21 | (15) | 0 | (0) | 21 | (38) |
| Death from any cause (n | 139 | (100) | 15 | (11) | 0 | (0) | 15 | (27) |

(%))

^{a}BSA = body surface area; CKD-EPI = Chronic Kidney Disease-Epidemiology Collaboration; eGFR = estimated glomerular filtration rate; IQR = interquartile range; RRT = renal replacement therapy; SD = standard deviation.
^{b}Values are given as n (%) for categorical variables, or mean ± SD for normally distributed continuous variables, unless otherwise indicated. Median [IQR] values are presented for continuous variables that are not normally distributed.
^{c}Composite endpoint: ≥40% decrease in CKD-EPI eGFR, doubling of serum creatinine, renal replacement therapy, or mortality.
^{d}Slope of CKD-EPI eGFR was calculated only for individuals with 3 or more eGFR values over at least 1 year.

1. A method for determining the prognosis of chronic kidney disease (CKD) in a patient suffering therefrom, said method comprising measuring the amount of complement 3a des-arginine (C3a-desArg) in an ex vivo sample of said patient.

2. The method according to claim 1, wherein a decreased level of C3a-desArg in a sample of a subject compared to a reference value is indicative of an adverse outcome.

3. The method of claim 1, said method further comprising:
(a)(i) measuring the amount of one or both of soluble tumor necrosis factor receptor 1 (sTNFR1) and neutrophil gelatinase-associated lipocalin (NGAL) in an ex vivo sample of said patient, and
(b)(ii) establishing the significance of the level of the biomarkers by inputting each of the biomarker concentration values into a statistical methodology to produce an output value that indicates CKD prognosis for the patient.

4. The method of claim 3, wherein an increased level of sTNFR1 in a sample of a subject compared to a reference value is indicative of an adverse outcome.

5. The method of claim 3, wherein an increased level of NGAL in a sample of a subject compared to a reference value is indicative of an adverse outcome.

6. The method according to claim 3, wherein an increased level of sTNFR1 in a sample of a subject compared to a reference value in combination with an increased level of NGAL in a sample of a subject compared to a reference value and a decreased level of C3a-desArg in a sample of a subject compared to a reference value is indicative of an adverse outcome.

7. The method according to claim 1, wherein the patient sample is selected from a serum, plasma or whole blood sample.

8. The method according to claim 1, wherein the patient is suffering from CKD stage 3 or above.

9. The method of claim 1, wherein an adverse outcome consists of >40% decline in CKD-EPI eGFR, doubling of serum creatinine, need for renal replacement therapy, or death.

10. The method of claim 1, wherein the statistical methodology used is logistic regression, decision trees, support vector machines, neural networks, random forest or another machine learning algorithm.

11. A method of screening a sample from a subject to determine prognosis of Chronic Kidney Disease (CKD), the method comprising contacting the sample with a substrate comprising a probe which binds specifically to soluble tumor necrosis factor receptor 1 (sTNFR1), a probe which binds specifically to neutrophil gelatinase-associated lipocalin (NGAL) and a probe which binds specifically to complement 3a des-arginine (C3a-desArg).

12. The method according to claim 11, wherein the subject is suffering from CKD stage 3 or greater.

13. A method for determining the prognosis of chronic kidney disease (CKD) in a patient suffering therefrom, said method comprising measuring the amount of NGAL and sTNFR1 in an ex vivo sample of said patient.
